(19) United States
(12) Patent Application Publication
(10) Pub. No.: US 2018/0111176 A1
(43) Pub. Date: Apr. 26, 2018

(54) INTEGRATED COLLECTION OF INFECTIOUS WASTE AND DISPOSAL THEREOF

(71) Applicant: AEMERGE LLC, Fortville, IN (US)

(72) Inventors: Landon C.G. MILLER, Fortville, IN (US); Scott BEHRENS, Fortville, IN (US)

(21) Appl. No.: 15/557,392

(22) PCT Filed: Mar. 11, 2016
(86) PCT No.: PCT/US2016/022061

§ 371 Date: Sep. 11, 2017

Related U.S. Application Data
(60) Provisional application No. 62/132,314, filed on Mar. 12, 2015.

(51) Int. Cl. B09B 3/00; C10B 53/07; A61L 9/16; B02C 19/00; B65G 67/30; A61B 50/36

(52) U.S. Cl. CPC B09B 3/0075; B09B 3/0083; C10B 53/07; A61L 9/16; B65G 15/28; B65G 67/30; A61B 50/36; A61L 2209/14; B02C 19/0075

(57) ABSTRACT

A system for treating infectious medical waste is provided that includes a collection infrastructure, handling, and a shredding sub-system for feeding partially processed waste to an oxidizer to eliminate potential airborne infectious waste prior to transforming the medical waste into useful co-products. Waste generators of potentially infectious medical waste include, but is not limited to hospitals, medical offices, dental offices, clinics, laboratories, research facilities, veterinarian offices, and emergency medical service providers. The medical waste is collected from the waste generators, transported to a processing site in sealed containers, and transformed into value-added products including hydrocarbon based gases, hydrocarbon-based liquids, and carbonized material in a system proceeded by a negative pressure region and having as a transformative element an anaerobic carbonizer.

### CROSS REFERENCE TO RELATED APPLICATIONS

This application claims priority benefit of U.S. Provisional Application Ser. No. 62/132,314 filed 12 Mar. 2015; the contents of which are hereby incorporated by reference.

### FIELD OF THE INVENTION

The present invention in general relates to a system for treating infectious waste; and in particular, to a medical waste collection infrastructure that is integrated with a handling and shredding sub-system with a built-in oxidizer to eliminate potential airborne infectious waste prior to transforming the medical waste into useful co-products, including hydrocarbon based gases, hydrocarbon-based liquids, and carbonized material in a system proceeded by a negative pressure region and having as a transformative element an anaerobic carbonizer.

### BACKGROUND OF THE INVENTION

Infectious medical waste is generated in the research, diagnosis, treatment or immunization of human beings or animals and has been, or is likely to have been contaminated by organisms capable of causing disease. Infectious medical waste includes items such as: cultures and stocks of microorganisms and biologicals; blood and blood products; pathological wastes; syringe needles; animal carcasses, body parts, bedding and related wastes; isolation wastes; any residue resulting from a spill cleanup; and any waste mixed with or contaminated by infectious medical waste. Facilities which generate infectious medical waste include: hospitals, medical offices, dentists, clinics, laboratories, research facilities, veterinarians, ambulance squads and emergency medical service providers, and the like. Infectious medical waste is even generated in private homes by home health care providers and individuals, such as diabetics, who receive injections at home.

Before infectious medical waste can be disposed of, the waste must be sterilized. Traditional sterilization methods include: incineration; steam treatment or autoclaving; and liquid waste may be disposed of in approved sanitary sewers. More recently developed methods that have been developed include microwave irradiation and use of various chemical washes. Transforming waste from a liability to an asset is a high global priority. Currently employed technologies that rely on incineration require disposal of waste while requiring scrubbers and other pollution controls to limit gaseous and particulate pollutants from entering the environment during incineration. It is also of note that incineration leaves useable energy in the waste generated. Incomplete combustion associated with conventional incinerators and the complexities of operation in compliance with regulatory requirements often mean that waste which would otherwise have value through processing is instead sent to a landfill or incinerated off-site at considerable expense. As medical waste often contains appreciable quantities of synthetic polymers including polyvinyl chloride, incineration of medical waste is often accompanied by release of ClOₓ, SOₓ, and NOₓ air pollutants that must be scrubbed from the emitted gases. Alternatives to incineration have met with limited success owing to complexity of design and operation outweighing the value of the byproducts from waste streams. Thus, the existing methods of disposing of infectious waste neither create energy nor usable byproducts to justify replacement of conventional medical waste incineration systems.

While there have been many advances in the treatment and disposal of infectious waste, there still exists a need for systems and methods for the safe collection, transfer, and treatment of infectious waste that maximize the economic return from the treated waste while enhancing handling safety and environmental controls.

### SUMMARY OF THE INVENTION

A system for treating infectious waste is provided with an unloading subsystem configured to remove one or more containers of infectious waste from a delivery transport. A belt conveyor of the unloading system extends from the exterior of a sealed enclosure to the interior of the sealed enclosure. A shredder in the interior of the sealed enclosure is fed a supply of waste from the belt conveyor. An oxidizer in fluid communication with the sealed enclosure destroys any airborne infectious matter from the sealed enclosure. A feed conveyor then transfers the shredded material from the shredder to a carbonizer.

A method of medical waste disposal includes supplying one or more carts each to a waste generator sites for holding one or more containers of infectious waste. The one or more carts are collected from the waste generator sites and are loaded on a transport vehicle. The transport vehicle conveys the carts to a centralized facility for treating infectious waste through a carbonization process.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1A is a perspective view of a waste holding cart holding barrels containing waste in accordance with an embodiment of the invention;

FIG. 1B is a perspective view of a waste holding cart holding boxes containing waste in accordance with an embodiment of the invention;

FIG. 2 is a detailed block diagram of the unloading subsystem that is part of an infectious waste treatment system as described further in FIG. 3 according to an embodiment of the invention;

FIG. 3 is a block diagram of an infectious waste treatment system according to an embodiment of the invention;

FIG. 4 is a side section view depicting an encapsulated shredding and infectious matter escape prevention sub-system according to an embodiment of the invention;

FIG. 5 is an oxidizer adapted for use with embodiments of the invention;

FIGS. 6A-6D are perspective pictorial views of the infectious waste treatment system as described in FIGS. 2-5 according to an embodiment of the invention;

FIG. 7 is a flowchart of a method for collecting waste material according to an embodiment of the invention; and

FIG. 8 illustrates a detailed block diagram of a carbonizer of FIG. 3 with a thermal oxidizer and thermal recovery system in accordance with embodiments of the invention.

### DETAILED DESCRIPTION OF THE INVENTION

The present invention has utility as a system for treating infectious waste. Through inclusion of a medical waste collection infrastructure, handling, and a shredding sub-system for feeding partially processed waste to an oxidizer to eliminate potential airborne infectious waste prior to transforming the medical waste into useful co-products, the aforementioned limitations of the prior art have been overcome. According to the present invention, medical waste is collected from waste generators, transported to a processing site, and transformed into value added products including hydrocarbon based gases, hydrocarbon-based liquids, and carbonized material in a system proceeded by a negative pressure region and having as a transformative element an anaerobic carbonizer. With medical waste as a feedstock for the production of valuable products, the present invention provides an economically viable and environmentally more responsible alternative to collecting waste from waste producers or generators with traditional methods of medical waste treatment. A waste generator of potentially infectious medical waste include, but is not limited to hospitals, medical offices, dental offices, laboratories, research facilities, veterinarian offices, and emergency medical service providers.

Embodiments of the invention provide the infrastructure and a method for safely and efficiently collecting medical waste from waste generators for further processing and environmentally responsible disposal. Embodiments of the inventive method provide carts to waste generators for collecting and storing waste products in containers such as conventional waste drums and boxes. The carts are collected by a waste disposal vehicle for transport to a waste processing plant. Examples of waste collection containers may include fiber based barrels and plastic lined cardboard boxes. The use of standardized carts and containers allows for consolidation of waste in the various generator facilities. During a waste collection visit the filled carts are rolled or loaded onto trucks and delivered to a waste treatment facility. In a specific embodiment individual loads from smaller collection trucks are consolidated into semi-trailers or train cars for transport to a treatment facility. The use of standardized carts and consolidation of waste collection may allow for the reduction of the number processing plants in a geographic area.

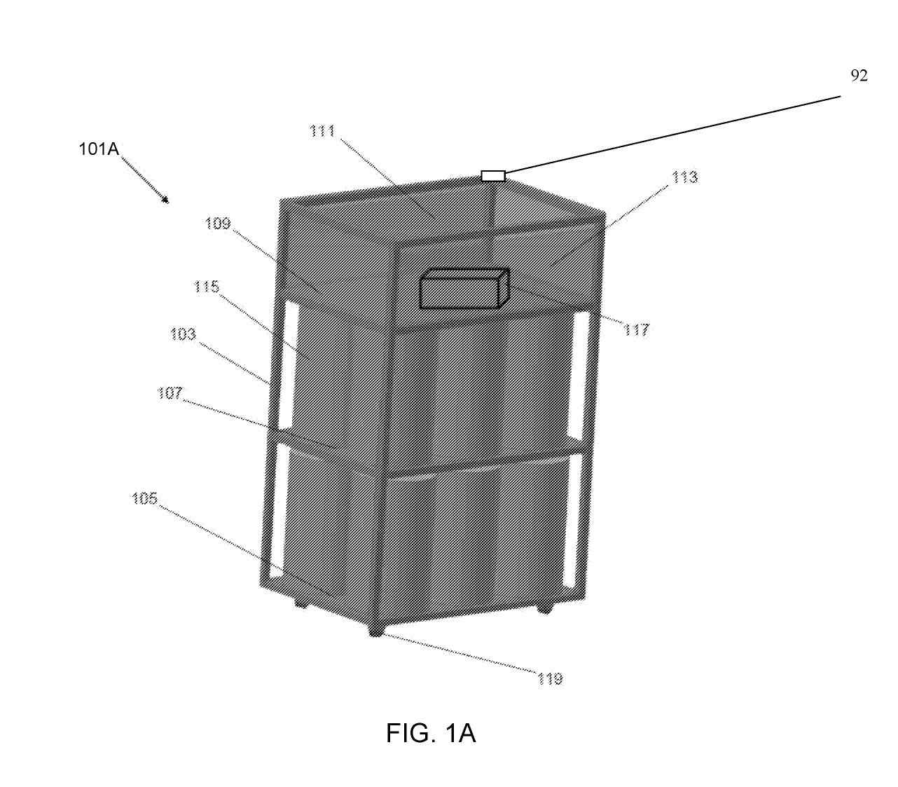

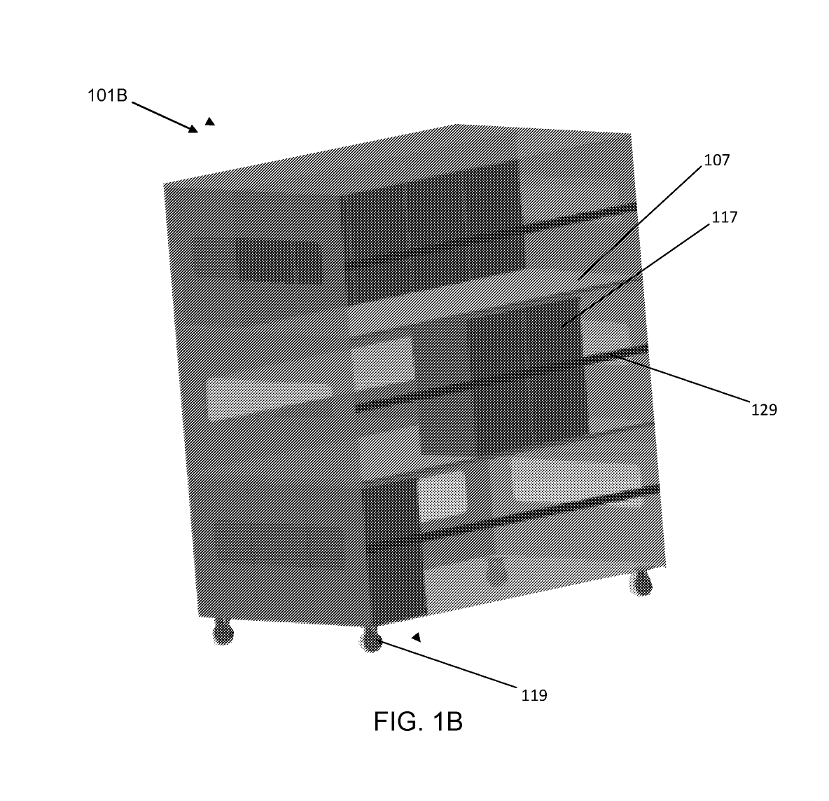

Referring now to the figures, embodiments of an inventive infectious waste collection and processing system are described. FIG. 1A is a perspective view of a waste holding cart 101A in accordance with an embodiment of the invention. The cart 101A has a support frame 103 with wheels 119 mounted to the bottom of a floor shelf 105, one or more middle shelves 107 above the floor shelf 105, and a top shelf 109. The spacing between the floor shelf 105 and amongst the one or more middle shelves 107 and the top shelf 109 may be configured to hold barrels or drums 115. In a specific embodiment, odd sized small containers (for example, containing sharps, or used needle containers) and plastic lined cardboard boxes 117 may be held in a top bin 111 formed with side walls 113 and the top shelf 109. One or more sensors 92 on a cart 101A or 101B may notify the waste disposal company when the cart 101A or 101B is full. A signal from a sensor 92 is readily communicated to alert a transport vehicle or operator thereof to schedule a pick-up, else the waste generator is so alerted. An optional top door (not shown) may seal the top bin 111 of the cart 101A. FIG. 1B is a perspective view of a waste holding cart 101B in accordance with an embodiment of the invention that is configured to hold plastic lined cardboard boxes 117 containing waste. The cart 101B may have similar overall dimensions to the barrel holding cart 101A. Retaining bars 129 keep the boxes 117 from falling off the cart 101B.

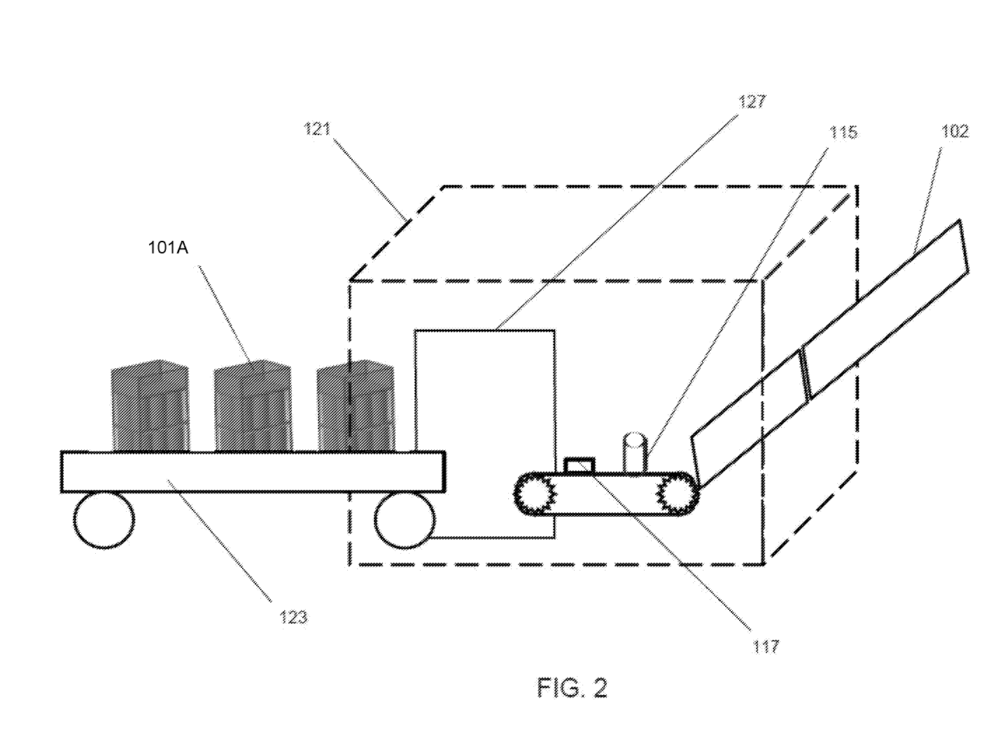

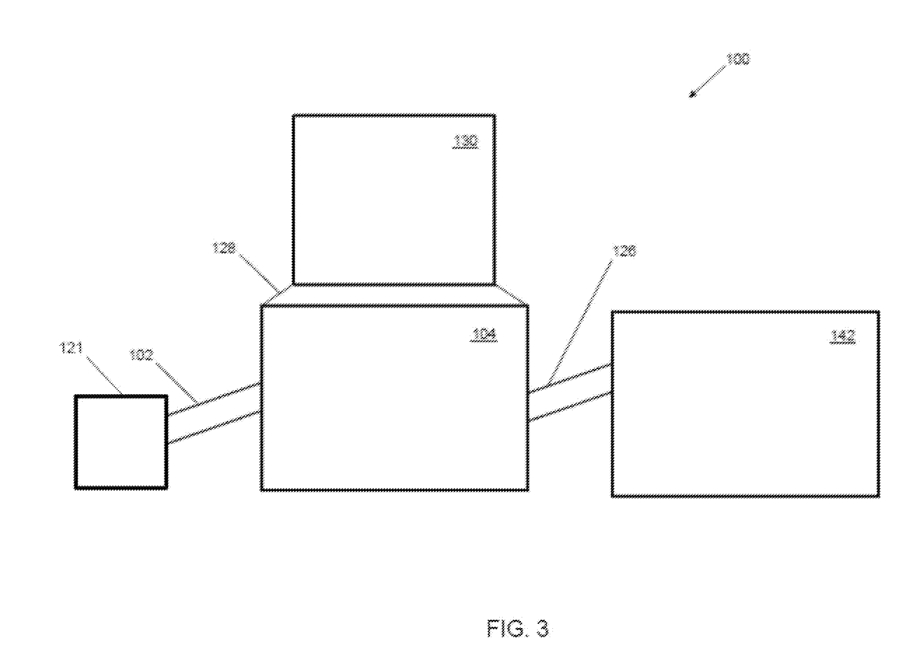

FIG. 2 is a detailed block diagram of the unloading subsystem 121 that is part of an infectious waste treatment system 100 as described further in FIG. 3 according to an embodiment of the invention. The unloading subsystem 121 (shown as dotted lines to expose the inner components) is configured to accept carts 101 that are loaded on a transport vehicle such as a truck trailer or rail car 123. An automated handling system 127 is configured to accommodate the carts 101 for offloading without the need for workers to touch the contents of the cart 101. Unloading techniques used may include tipping the cart 101, or using a plunger, to push the barrels 115, boxes 117, and odd sized containers onto an integrated conveying system 102 that feeds the cart waste contents for further processing in the infectious waste treatment system 100 as described further in FIGS. 3-5. In a specific embodiment, machine vision and robotic arms may be used to offload the carts 101 and place the contents on conveyor 102. The use of automation reduces costs, and improves infection safety by using the essentially no touch process. The containers (115, 117) may have radio frequency identification (RFID) tags or indicia (such as barcodes) that are read to, for example, confirm receipt of waste products from a particular waste generator. The identified waste may also be weighed for billing purposes as the waste is offloaded from the cart 101.

FIG. 3 is a block diagram of an infectious waste treatment system 100 according to an embodiment of the invention. An encapsulated shredding and infectious matter escape prevention sub-system 104 encloses a shredder in a negative pressure sealed environment that acts to contain residue and contaminants from escaping into the environment during the shredding operation. The infectious waste is loaded into the sub-system 104 via belt conveyor 102 that connects the unloading subsystem 121 to the infectious matter escape prevention sub-system 104. The belt conveyor 102 introduces the infectious or contaminated waste in bags or containers (115, 117) into the subsystem 104. An oxidizer 130 destroys any airborne infectious matter that exits through hood 128 at the top of the sub-system 104.

As used herein an oxidizer is defined to also include a thermal oxidizer and catalytic oxidizer; such systems are commercially available and in widespread usage.

Feed conveyor 126 transfers the shredded material from the sub-system 104 to the carbonizer 142. It is appreciated that feed conveyor 126 optionally also includes augers, shuttle bins, and other conventional devices to transit shredded material; the inclusion of such devices depending on factors such as desired throughput, shred size, and shred content.

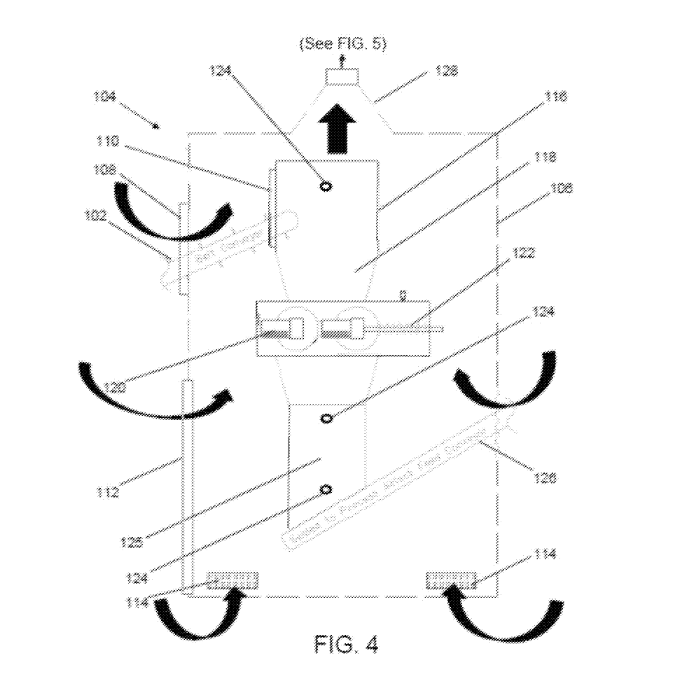

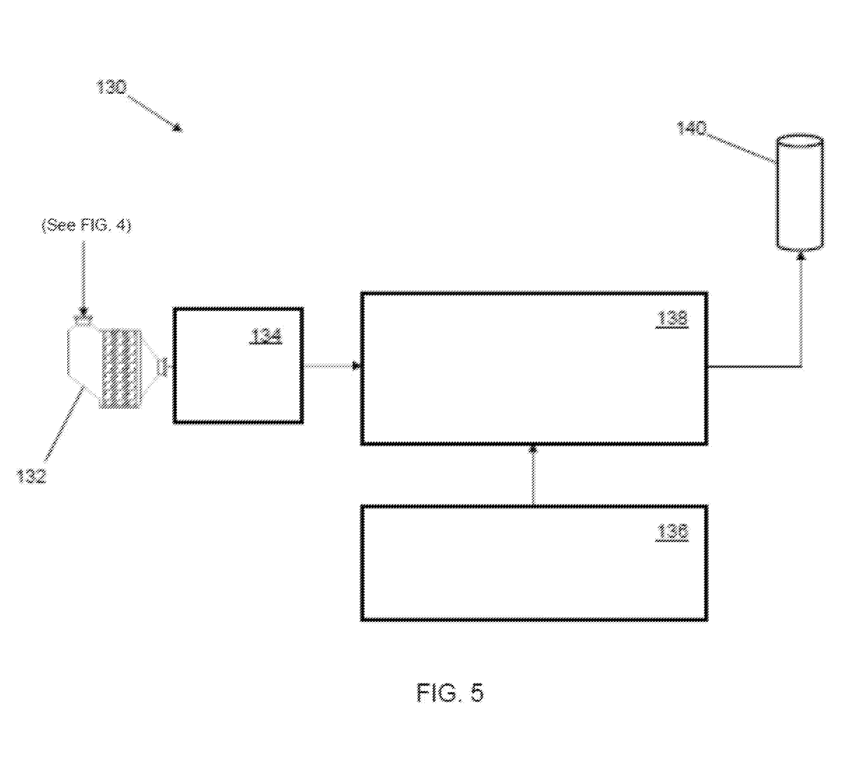

FIG. 4 is a side section view depicting the encapsulated shredding and infectious matter escape prevention sub-system 104. The dotted lines represent the containment walls 106 that enclose the shredder 116. The enclosure of the sub-system 104 is maintained at a negative pressure to draw in air (as opposed to expelling air) as represented by the arrows into the vents 114, as well as into the exterior flap 108 that permits containerized waste to enter the sub-system 104 via the belt conveyor 102, and other openings such as for the feed conveyor 126 and service door 112. It is appreciated that in certain inventive embodiments the incline of the feed conveyor 126 is from 1 to 10 degrees, while in other embodiments from 2 to 5 degrees to promote gravity feed and inhibit conveyor mechanism fouling. The exterior flap 108 is readily formed of rubberized materials, polymeric sheeting, as well as metals. Service door 112 is provided in some inventive embodiments to allow service workers to enter the enclosure. It is appreciated that a service person may be required to wear protective clothing and a filter mask. In a specific embodiment the service door 112 may be a double door airlock, where only one door is open at a time to minimize the escape of contaminants into the environment. In still other embodiments, the air handling system modifies operation during opening of the service door 112 to maintain a negative pressure during opening to inhibit airborne escape of potential pathogens. Hopper flap 110 acts to allow containerized waste to enter the hopper 118 of the shredder 116, while also acting as a seal around the belt conveyor 102. The hopper flap 110 is readily formed of rubberized materials, polymeric sheeting, as well as metals. At the bottom of the hopper 118, an auger 122 that is driven by one or more motors 120 shreds the waste. In an embodiment the motors 120 may be variable frequency drive (VFD) motors. The shredded material is accumulated in a process airlock 125 that supplies material to a feed conveyor 126. Levels and presence of material within the hopper 118 and the process airlock 125 are controlled via sensors 124. In a specific embodiment the sensors 124 are through beam sensors (TBS). Feed conveyor 126 is sealed to the process airlock 125, and transports the shredded material from the sub-system 104 to the carbonizer 142. Hood 128 collects airborne contaminants for introduction into the oxidizer (TO) 130.

FIG. 5 is a block diagram of an oxidizer 130 adapted for use with embodiments of the invention that acts as a fume incinerator for the containment room of sub-system 104. Large particle screener 132 filters out particles from the exhaust stream of airborne contaminants. A filter differential sensor may be employed to detect when a filter is clogged and requires replacement. A blower 134 draws in the exhaust stream and blows the exhaust stream into the combustion tube 138. A gas supply 136 supplies fuel for burners in the combustion tube 138. In specific embodiments, the oxidizer 130 is run on a mixture of natural gas and reaction-produced carbonization process gases re-circulated to transform the heat through the use of either conventional steam boilers or to Organic Rankin Cycle strategies to operate electrical turbine generators, or in the alternative, to reciprocating engine driven generators, and thereby generate the heat needed to produce power while also operating the carbonization process in the carbonizer 142. This heat capture produces more waste heat than is used to heat water and generate steam for turbines or steam reciprocating engines. This heat in some inventive embodiments is used to preheat feedstock or for other larger process purposes. The pre-processing heating system preheats feedstock material prior to entering the reactor tube to both reduce moisture and improve overall system yield. Roof exhaust stack 140 vents cleaned exhaust to the environment.

An apparatus for anaerobic thermal transformation processing is a carbonizer 142 that converts input infectious medical waste into useable products such as bio-gas; bio-oil; carbonized materials; non-organic ash. Such a carbonizer 142 is detailed in U.S. Pat. No. 8,801,904; the contents of which are incorporated herein by reference.

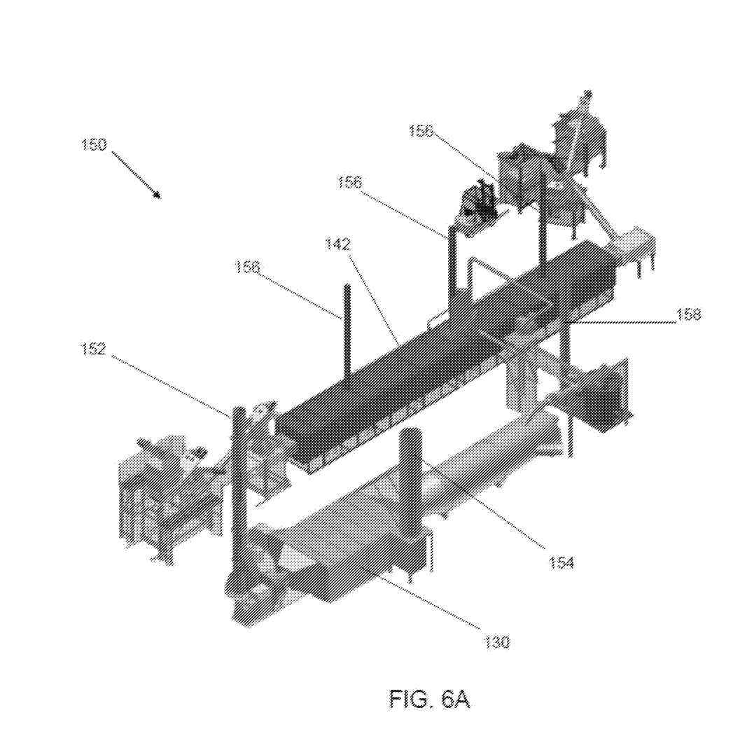

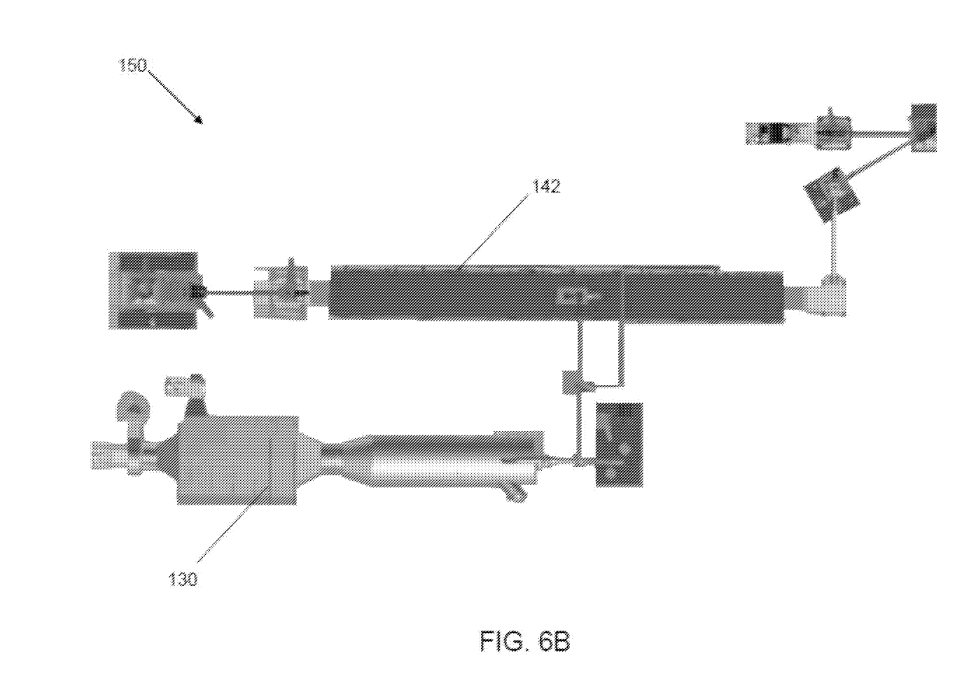

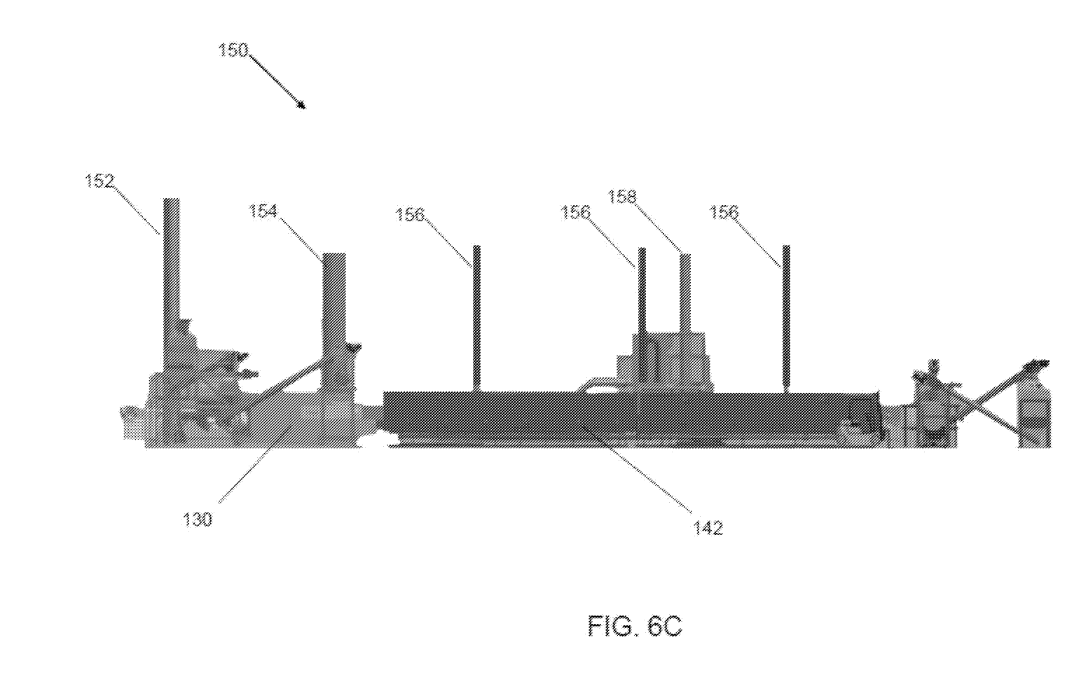

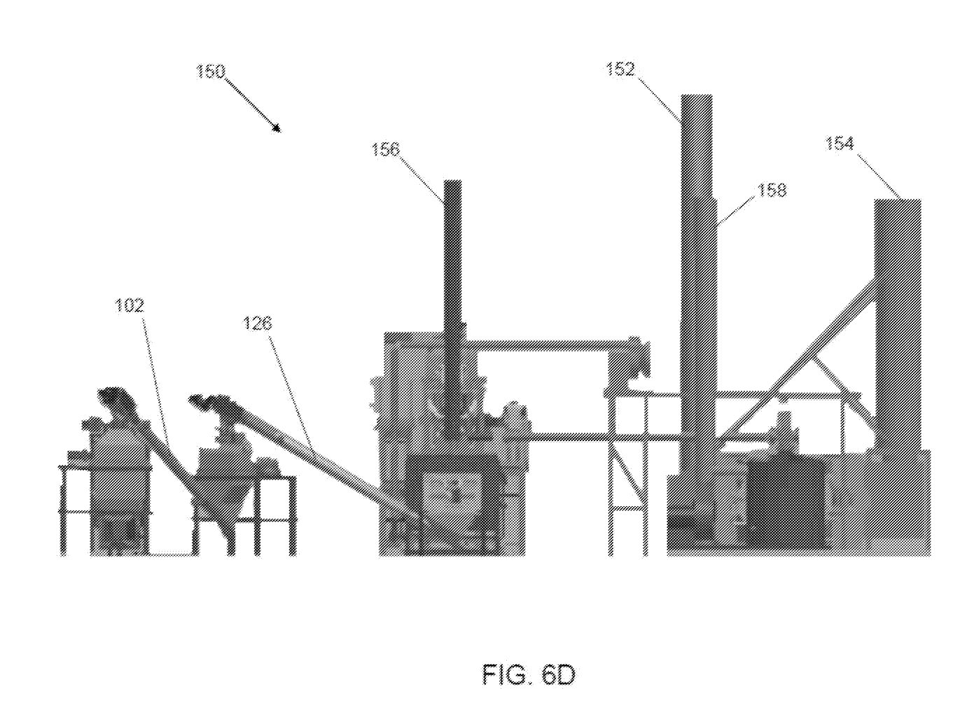

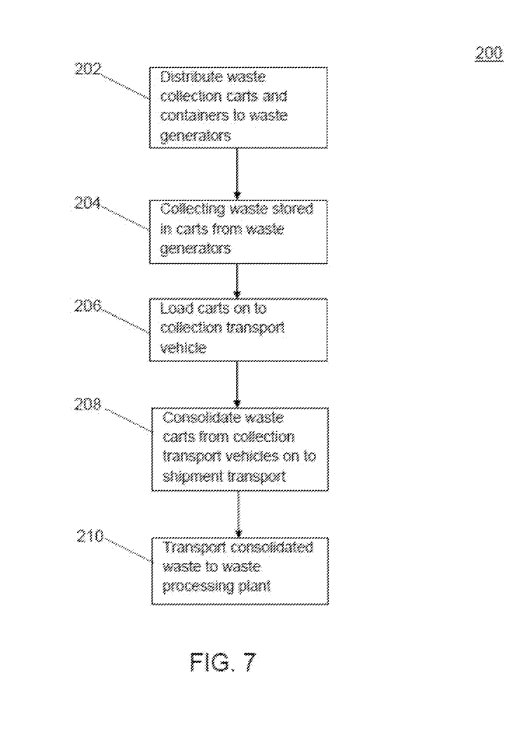

FIGS. 6A-6D are perspective pictorial views of the infectious waste treatment system 150 with components as described in FIGS. 2-5 according to an embodiment of the invention. Exhaust stacks visible in FIGS. 6A-6D include oxidizer (thermal) exhaust 152, heat exchanger exhaust 154, furnace natural gas burner exhaust 156, and emergency vent exhaust 158. FIG. 7 is a flowchart of a method 200 for collecting waste material from generators using the carts 101 according to an embodiment of the invention. Waste carts are distributed to waste generators with containers (115, 117) for storing waste (block 202). Waste carts are collected from waste generators on a fixed interval, or on demand from the client. In certain inventive embodiments, sensors on a cart 101A or 101B may notify the waste disposal company when the cart 101A or 101B is full (block 204). Carts are loaded on to a collection transport vehicle for transport to a local holding area (step 206). The carts in local holding area are consolidated on to a larger shipment transport for shipment to processing plant (step 208). The consolidated waste load is transported to processing plant.

The present is further contemplated as integrating an infectious waste disposal system as detailed in Patent Cooperation Treaty Application PCT/US16/13067; the contents of which are hereby incorporated by reference.

### EXAMPLES

### Example 1—Transfer Station to Treatment Facility Delivery System—Drum Cart

A cart as described with respect to an embodiment of FIG. 1A is configured to hold twelve 210 litter fiber drums with 1.5-mil polyethylene liners. The average fiber drum weight is 33 kg maximum capacity at 160 kgs/m³ of medical waste with total weight of 41 kgs. Drums are loaded onto portable drum carts and safely secured into position for transport. A 16 meter long trailer of a transport vehicle can hold up to 18 carts that hold a total of 216 drums with a total weight of 8,820 kgs. The trailer may be manually unloaded in less than three hours based on an estimate of ten minutes per cart. The two layers of stored drums effectively doubles transfer station and treatment center floor space storage, where each cart provides sixty square feet of equivalent storage space. The drum cart may be manually rolled into unloading station at the treatment center where an automated system pushes the drums into the shredder pre-feed system.

### Example 2—Transfer Station to Treatment Facility Delivery System—Box Cart

A cart as described with respect to an embodiment of FIG. 1B is configured to hold forty five boxes, the boxes each measuring 45×45×90 cm with 1.5-mil polyethylene liners. In a specific embodiment the boxes are made of cardboard. The average box weight is 27 kgs maximum capacity at 160 kgs/m³ of medical waste with total weight of 29 kgs. Drums are loaded onto portable box carts and safely secured into position for transport. A 16 meter long trailer of a transport vehicle can hold up to 11 carts that hold a total of 495 boxes with a total weight of 14,145 kgs. The trailer may be manually unloaded in less than two hours based on an estimate of ten minutes per cart. The three layers of stored boxes effectively doubles transfer station and treatment center floor space storage, where each cart provides 105 square feet of equivalent storage space. The box cart may be manually rolled into unloading station at the treatment center where an automated system pushes the drums into the shredder pre-feed system.

### Example 3 —Medical Waste Processing and Safe Thermal Conversion

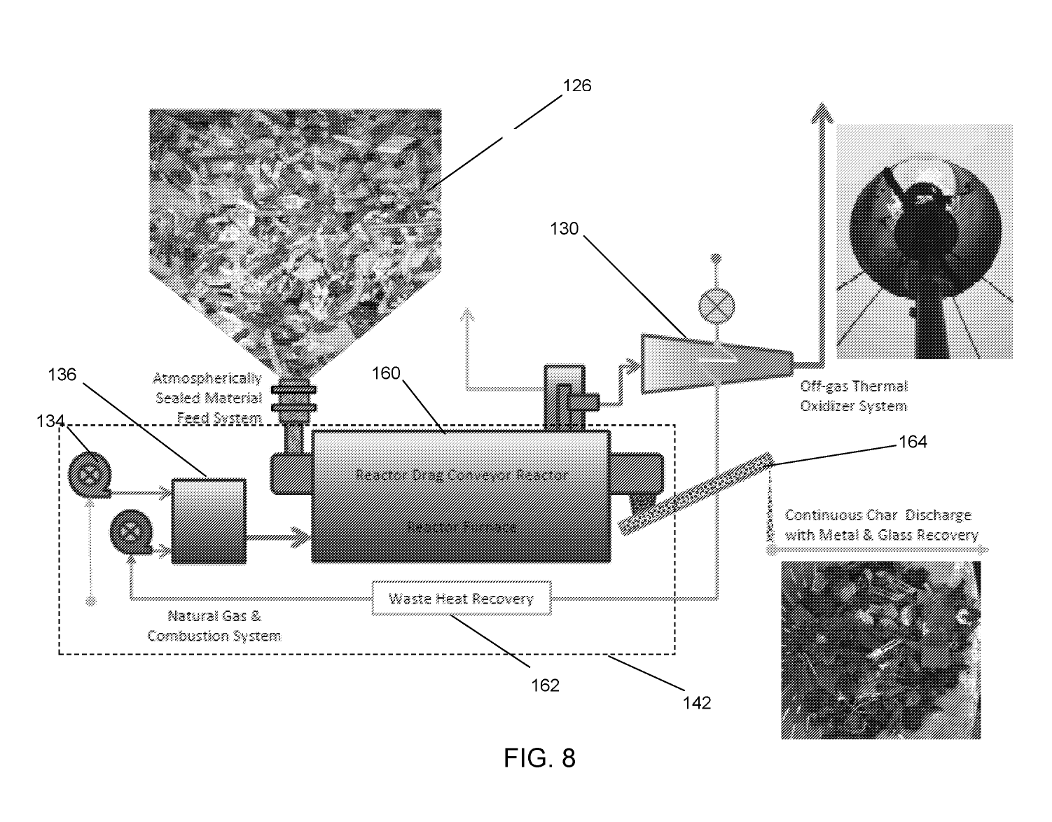

As described in conjunction with FIGS. 1-5 and in FIG. 8, a process for generating char and the recovery of metal and glass is as follows. A carbonizer 142 receives shredded waste material from a sealed feed material system 126 and the waste material is fed to a reactor furnace 160 that may in a specific embodiment be a drag conveyor reactor. Off-gas from the reactor furnace 160 is treated with a thermal oxidizer 130, and waste heat generated in the thermal oxidizer 130 is supplied via a waste heat recovery system 162 with a blower 134 into the reactor 160 along with supply gas 136 for the burners in the reactor 160. The reactor outputs a continuous char discharge 164 along with recoverable metal and glass. In a specific embodiment, the waste is heated at approximately 730° C. to 790° C. for 30 minutes in the reactor. The resultant organic waste (approximately 75% of output) is converted to gas phase and oxidized in the thermal oxidizer 130, and the remaining 25% of the output is char with recoverable metals and glass. Material processed according had the composition of the Table 1.

TABLE 1
Carbonized Output Material Analysis, where percentages are
total weight percent

| | | As | |
|---|---|---|---|
| Component | Method | Received | Dry |
| Moisture | ASTM D4442(A) | 1.65 | 0 |
| Ash* | ASTM D1102 | 40.30 | 40.98 |
| Volatile matter | ASTM D3175 | 12.81 | 13.02 |
| Fixed carbon | ASTM D3172 | 45.24 | 46.00 |
| Sulfur | ASTM D4239(A) | 0.49 | 0.50 |
| Carbon | ASTM D5573 | 54.23 | 55.14 |
| H + N + O | ASTMD5573 | 3.38 | 3.38 |

*major ash constituents in decreasing percentage as oxides: silicon, calcium, iron, aluminum, titanium, sodium, magnesium, sulfur, phosphorus, undetermined, potassium, manganese, barium, and strontium. The above oxides totaled more than 98 weight percent of the ash.

As a person skilled in the art will recognize from the previous detailed description and from the figures and claims, modifications and changes can be made to the preferred embodiments of the invention without departing from the scope of this invention defined in the following claims.

1. A system for treating infectious waste, the system comprising:
an unloading subsystem configured to remove one or more containers of infectious waste from a delivery transport;
a sealed enclosure;
a shredder within said sealed enclosure;
a belt conveyor from said unloading subsystem to said sealed enclosure to supply said waste, where said belt conveyor runs from the exterior of said sealed enclosure to said shredder;
an oxidizer in fluid communication with said sealed enclosure adapted to sterilize airborne infectious matter from said sealed enclosure; and
a feed conveyor for transfer of shredded material from said shredder to a carbonizer.

2. The system of claim 1 further comprising a cart supporting said one or more containers.

3. The system of claim 2 wherein said unloading subsystem is configured to remove said cart holding said one or more containers of infectious waste from a delivery transport, where said cart further comprises:
a support frame with a set of wheels mounted to a bottom surface of a floor shelf;
one or more middle shelves above said floor shelf; and
a top shelf, a spacing between any two of said floor shelf, said one or more middle shelves and said top shelf configured to hold a set of barrels or drums.

4. The system of claim 1 further comprising a rubberized exterior flap adapted to permits containerized and bagged waste to enter said sealed enclosure via said belt conveyor.

5. The system of claim 1 wherein said oxidizer further comprises a large particle screener adapted to filter out particles from said airborne infectious matter.

6. The system of claim 1 wherein said oxidizer further comprises a blower adapted to draw said airborne infectious matter into a combustion tube.

7. The system of claim 1 wherein said sealed enclosure further comprises a hood adapted to collect said airborne contaminants and in communication with said thermal oxidizer.

8. The system of claim 1 further comprising a roof exhaust stack to vent cleaned exhaust.

9. The system of claim 1 wherein said feed conveyor is inclined.

10. The system of claim 1 wherein said sealed enclosure is maintained at a negative pressure.

11. The system of claim 1 wherein said one or more containers is a fiber based barrel or a plastic lined cardboard box.

12. The system of claim 1 wherein said one or more containers further comprise a radio frequency identification (RFID) tag or indicia.

13. The system of claim 1 wherein said unloading subsystem further comprises a tipping mechanism to remove said one or more containers of infectious waste from said delivery transport.

14. The system of claim 1 wherein said unloading subsystem further comprises a plunger to push said one or more containers of infectious waste from said delivery transport.

15. The system of claim 1 wherein said unloading subsystem further comprises machine vision and a robotic arm to remove said one or more containers of infectious waste from said delivery transport.

16. The system of claim 1 wherein said oxidizer is a thermal oxidizer.

17. The system of claim 1 wherein said oxidizer further comprises a gas supply in communication with a set of burners in a combustion tube.

18. (canceled)

19. The system of claim 1 wherein said shredder further comprises a hopper and a process airlock in which shredded wasted material accumulates and is then transferred to said feed conveyor.

20. (canceled)

21. The system of claim 20 wherein said one or more sensors are through beam sensors.

22. A method of medical waste disposal comprising:
supplying one or more carts to each to a plurality of waste generators, each of said one or more carts for holding one or more containers of infectious waste;
collecting said one or more carts from said a plurality of waste generators;
loading said one or more carts on a transport vehicle; and
conveying said one or more carts to a centralized facility on said transport vehicle for treating infectious waste through a carbonization process.

23. The method of claim 22 further comprising consolidating said one or more carts from said transport vehicle with infectious waste from a plurality of transport vehicles prior to conveyance to said system for treating infectious waste.

24. The method of claim 22 wherein said collecting is conducted on a fixed interval of time or on demand from said series of waste generators.

25. The method of claim 22 wherein a set of sensors notify said transport vehicle or an operator thereof when to conduct said collecting.

26. The method of claim 22 wherein said centralized facility is a system of claim 1.
